(19) United States
(12) Patent Application Publication
(10) Pub. No.: US 2005/0209617 A1
(43) Pub. Date: Sep. 22, 2005

(54) Valvulotome

(76) Inventors: Paul Koven, St. Louis, MO (US); Joan Koven, St. Louis, MO (US); Dale Buchbinder, Baltimore, MD (US)

Correspondence Address: GREENSFELDER HEMKER & GALE PC, SUITE 2000, 10 SOUTH BROADWAY, ST LOUIS, MO, 63102, US

(21) Appl. No.: 11/071,973

(22) Filed: Mar. 4, 2005

Related U.S. Application Data
(60) Provisional application No. 60/550,710, filed on Mar. 5, 2004.

(51) Int. Cl. A61B 17/22

(52) U.S. Cl. 606/159

(57) ABSTRACT

A valvulotome device having retractable cutting blades and guide fins which permits continual centralizing, self-alignment of the cutting blades within the lumen of the vein is disclosed. The valvulotome device comprises a bifurcated assembly having a cutter head and guide head joined by a conduit member permitting communication of a catheter wire running longitudinally through the device. The cutter head and guide head each define a chamber adapted to receive the catheter wire therein. First and second wedge members are operatively engaged to the catheter wire and disposed inside a respective chamber of the cutter head and guide head which permit the surgeon to remotely manipulate the degree to which the cutting blades and guide fins emerge from the cutter head and guide head, respectively, as well as allow rotation of the device about its axis thereby increasing the maneuverability of the device as it is pulled through the vein.

### CROSS REFERENCE TO RELATED APPLICATIONS

This application claims priority from U.S. Provisional Patent Application Ser. No. 60/550,710, filed Mar. 5, 2004.

### FIELD OF THE INVENTION

The present invention relates to a medical device for the treatment of vascular disorders, and more particularly to a valvulotome for excising venous valves when performing in-situ vascular procedures, such as arterial bypass surgery.

### BACKGROUND OF THE INVENTION

Occasionally, as part of the medical treatment for distressed arteries in a patient's lower extremities, a bypass of an artery through its neighboring vein is made. This enables the vein to be used in place of the artery as the source of, and conduit for, blood pumped from the heart to the lower extremities. Unlike arteries, however, veins have internal one-way valves spaced periodically within the lumen of the vein which function to ensure that the flow of blood is directed back to the heart and does not reverse itself. These valves are comprised of tissue flaps disposed on the luminal wall inside the vein. The valves open when leg muscles contract to force blood to flow out of the lower extremities. The valves close when blood attempts to flow backwards after the leg muscles relax, thereby preventing a reverse flow of blood back through the vein. When a vein is recruited for use in an arterial bypass, it is crucial that the valves in the vein be removed, because they will otherwise impede the arterial flow if left in place.

There exist devices, called valvulotomes, for cutting and removing the venous valve from the luminal passageways of the vein. These devices are designed to be inserted into the vein and passed along the lumen over the section of the vein that is to participate in the arterial bypass. The valvulotome is provided with one or more blades which are disposed on the device in a position to cut the venous valve. It is, however, essential that the venous wall itself is not cut or damaged by the blade because that would jeopardize the effectiveness of the vein as a bypass vessel.

Prior art valvulotomes have been equipped with certain safeguards to help prevent inadvertent damage to the luminal walls of the vein. In some devices, the blades are configured to be retractable so that the valvulotome, with the blades in the closed position, can be initially inserted in the vein through the desired region and then be withdrawn, with the blades in an open position, to cut the valves. However, even a retractable blade can cause damage to the vein wall upon withdrawal of the valvulotome if the head of the device bearing the blades is not disposed centrally and evenly within the lumen of the vein. For instance, if the head of the valvulotome is off center within the lumen, one side of the vein wall may be cut into too deeply, while the other side of the vein wall may not even be engaged, thus leaving the valve on that side still intact in the vein.

Some valvulotome devices provide for a centering mechanism to ensure that the valvulotome blades are centrally disposed within the vein lumen during the excision. This centering mechanism may comprise an enlarged body portion spaced proximally to, and slightly apart, from the blades. This serves two primary purposes: 1) to provide an annular guide for centralizing the blades within the lumen of the vein, and 2) to spread the vein walls apart to a greater degree than the cutting radius of the blades to limit the reach of the blades to the valves and prevent the blades from contacting the vein walls themselves. However, for veins having changing diameters along the area of treatment, a centering mechanism having a fixed dimension may be counterproductive in the cutting operation. If the centering mechanism is substantially less than the diameter of the vein lumen, it will not effectively center the valvulotome. If the centering mechanism is substantially greater than the diameter of the vein lumen, it may draw the valve flaps out of cutting range of the blades.

Valvulotome devices are generally introduced into and through the vein by the use of catheters. While catheters are of extremely slender diameter and are quite flexible, a turn or bend in the vein, or branching of the vein, may place a kink in the catheter. If the valvulotome head is rigidly connected to the catheter, it may affect the ability of the blades to be centrally disposed within the vein lumen, thereby potentially causing an adverse effect on the excision procedure.

Therefore, there is a need in the art for a valvulotome device having adjustable cutting blades and an adjustable guide mechanism which permits continual centralizing, self-alignment of the cutting blades within the lumen of the vein.

### SUMMARY OF THE INVENTION

A primary object of the present invention is to provide a valvulotome device that permits continual, self-alignment of the cutting blades within the lumen of a vein.

Another object of the present invention is to provide a valvulotome device having adjustable cutting blades.

A further object of the present invention is to provide a valvulotome device having a bifurcated assembly comprising a distal body including a cutter head and a proximal body including a guide head that may be manipulated simultaneously by the user.

Another further object of the present invention is to provide a valvulotome device that permits remote manipulation and navigation of the device by a user through the vein of a patient.

Yet a further object of the present invention is to provide a valvulotome device having a guide mechanism that permits continual centralizing, self-alignment of the cutting blades of the device within the lumen of a vein.

In one embodiment, the present invention comprises a valvulotome device comprising a handle, a bifurcated assembly operatively engaged to handle having a distal portion including a cutter head, said cutter head having a plurality of retractable, radially oriented cutting blades and a proximal portion having a guide head, said guide head having a plurality of retractable, radially oriented guide fins, a catheter wire operatively engaged to cutter head and guide head through the handle and disposed within the bifurcated assembly, wherein said catheter wire permits remote manipulation of the cutter head and the guide head, operatively engaged to the catheter wire, first and second wedge members disposed inside the cutter head and guide head, respectively and in operative engagement with the plurality of cutter blades and plurality of guide fins, respectively, such that movement of each respective first and second wedge member controls the effective cutting diameter of the plurality of cutting blades and the effective diameter of the guide fins, respectively.

In another embodiment, the present invention comprises a method of use comprising: a) providing a valvulotome device comprising a handle, a bifurcated assembly operatively engaged to handle having a distal portion including a cutter head, said cutter head having a plurality of retractable, radially oriented cutting blades and a proximal portion having a guide head, said guide head having a plurality of retractable, radially oriented guide fins, a catheter wire operatively engaged to cutter head and guide head through the handle and disposed within the bifurcated assembly, wherein said catheter wire permits remote manipulation of the cutter head and the guide head, operatively engaged to the catheter wire, first and second wedge members disposed inside the cutter head and guide head, respectively and in operative engagement with the plurality of cutter blades and plurality of guide fins, respectively, such that movement of each respective first and second wedge member controls the effective cutting diameter of the plurality of cutting blades and the effective diameter of the guide fins, respectively; b) inserting the bifurcated body into the vein; and c) pulling the bifurcated body back through the vein such that the valves of the vein are cut.

In yet another embodiment, the present invention comprises a bifurcated assembly for use in a valvulotome device comprising a conduit member joined between a distal portion having a cutter head defining a chamber and a proximal portion having a guide head defining a chamber the head including a plurality of retractable radially oriented cutting blades, and the guide head including a plurality of retractable, radially oriented guide fins, wherein each of the cutter head and the guide head includes a wedge member disposed in each respective chamber and being adapted to operatively engage a catheter wire such that the diameters of cutting blades and guide fins can be adjusted by manipulation of the catheter wire.

### BRIEF DESCRIPTION OF THE DRAWINGS

FIG. 1 is an illustration of the valvulotome device showing the handle operatively engaged to the bifurcated assembly according to the present invention.

FIG. 2 is a front view of the bifurcated assembly with the blades and fins in the fully retracted position according to the present invention;

FIG. 2A is a front view of the bifurcated assembly with the blades and fins in the fully expanded position according to the present invention;

FIG. 3 is an exploded view of the bifurcated assembly according to the present invention;

FIGS. 4A-4C illustrate the retractable and expanding operation of the cutter head and guide head for the bifurcated assembly according to the present invention;

FIG. 5 is a perspective view of a cutting blade for the cutter head according to the present invention;

FIG. 5A is an enlarged view of the cutting blade according to the present invention;

FIG. 6 is a partial cross-sectional view of the guide head showing the swivel member and wedge member according to the present invention;

FIG. 7 is a side view of the handle according to the present invention;

FIG. 8 is a cross-sectional view of the handle taken along line 8-8 of FIG. 7 according to the present invention;

FIG. 9 is a cross-sectional view of the handle taken along line 9-9 of FIG. 7 according to the present invention;

FIG. 10 is a cross-sectional view of the handle taken along line 10-10 of FIG. 7 according to the present invention;

FIG. 11 is an exploded view of the handle according to the present invention;

FIG. 12 is a cross-sectional view showing the handle in the closed position according to the present invention;

FIG. 12A is an enlarged view of FIG. 12 according to the present invention;

FIG. 13 is a cross-sectional view showing the handle in the open position according to the present invention;

FIG. 13A is an enlarged view of FIG. 13 according to the present invention;

FIG. 14 is an orthogonal view of the wedge member according to the present invention;

FIG. 15 is an end view taken along line 15-15 of FIG. 2 with the cutting blades in their fully retracted position according to the present invention;

FIG. 15A is an end view taken along line 15A-15A of FIG. 2A with the cutting blades in their fully expanded position according to the present invention;

FIG. 16 is a cross-sectional view taken along line 16-16 of FIG. 2 with the guide fins in their fully retracted position according to the present invention; and

FIG. 16A is a cross-sectional view taken along line 16A-16A of FIG. 2A with the guide fins in their fully expanded position according to the present invention.

### DETAILED DESCRIPTION OF THE INVENTION

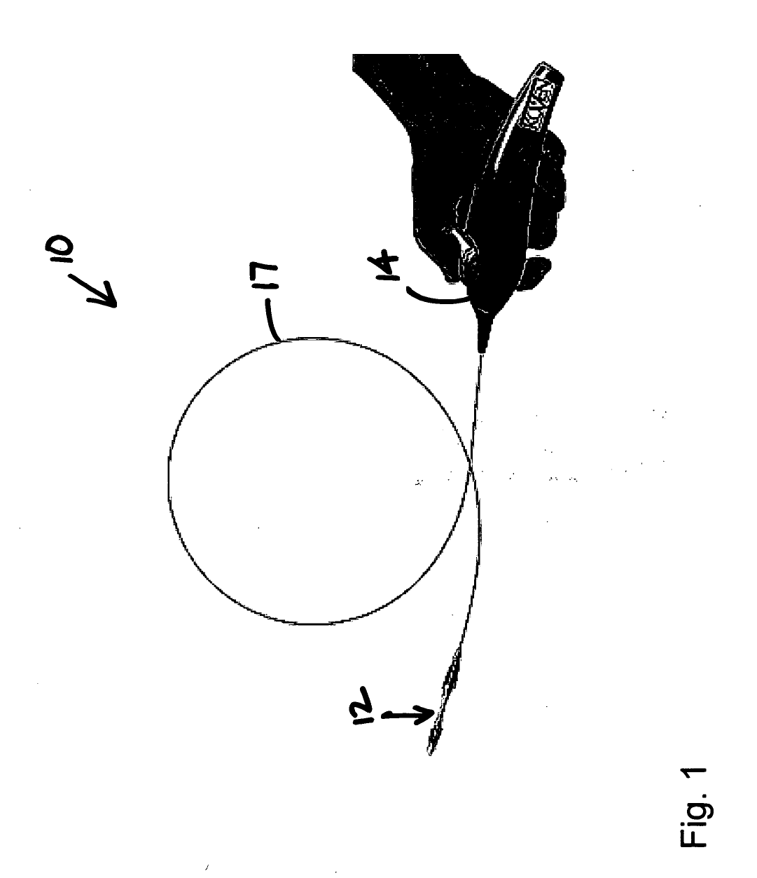

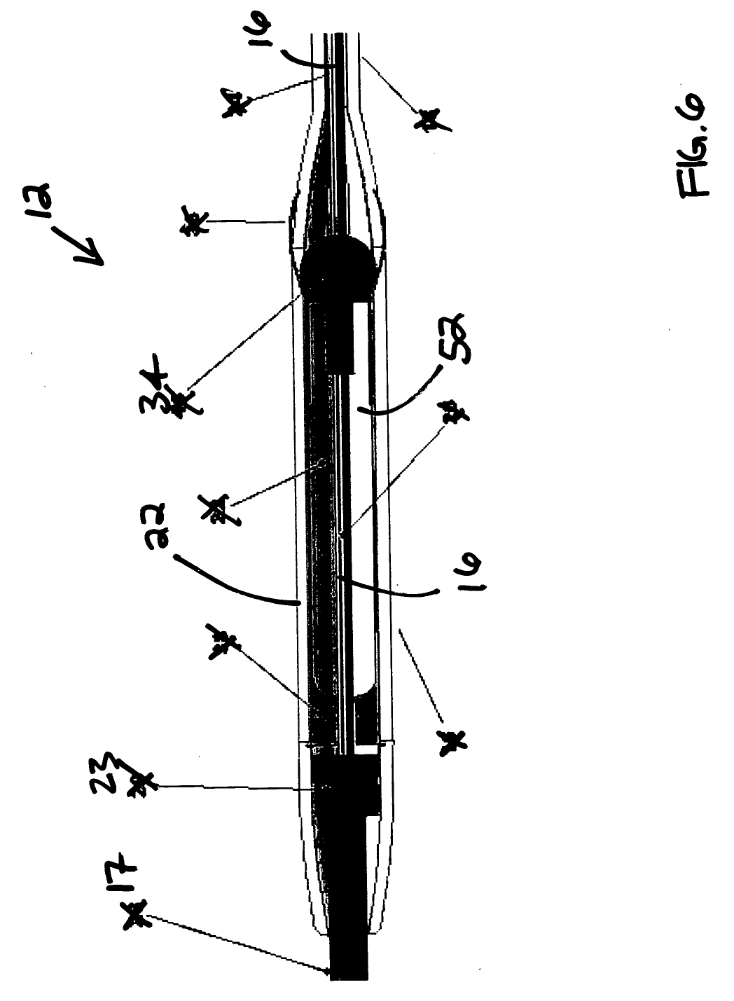

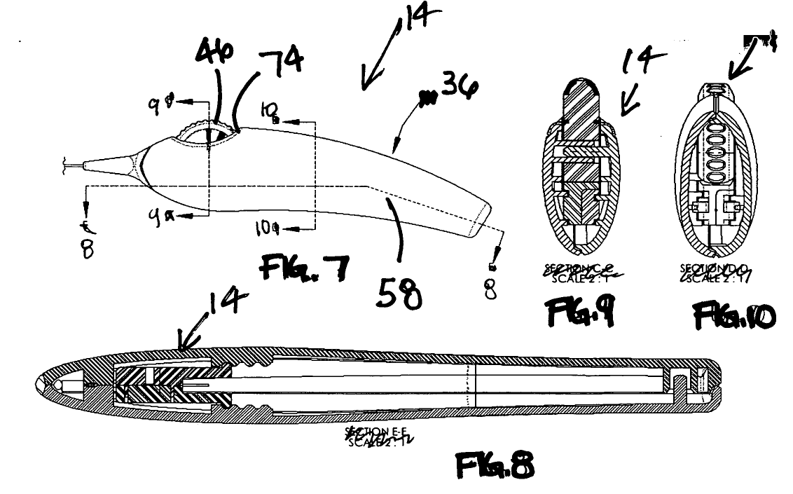

Referring to the drawings, a preferred embodiment of the valvulotome device according to the present invention is illustrated and generally indicated as 10 in FIGS. 1-16. The valvulotome device 10 comprises bifurcated assembly 12 operatively associated with a handle 14 through a catheter wire 16 encased inside the lumen of a flexible catheter 17. The bifurcated assembly 12 provides a means for cutting valves in a vein of a patient as well as a guide means for maintaining the cutting means in a continual centralizing, self-alignment within the lumen of the vein when operated by the handle 14.

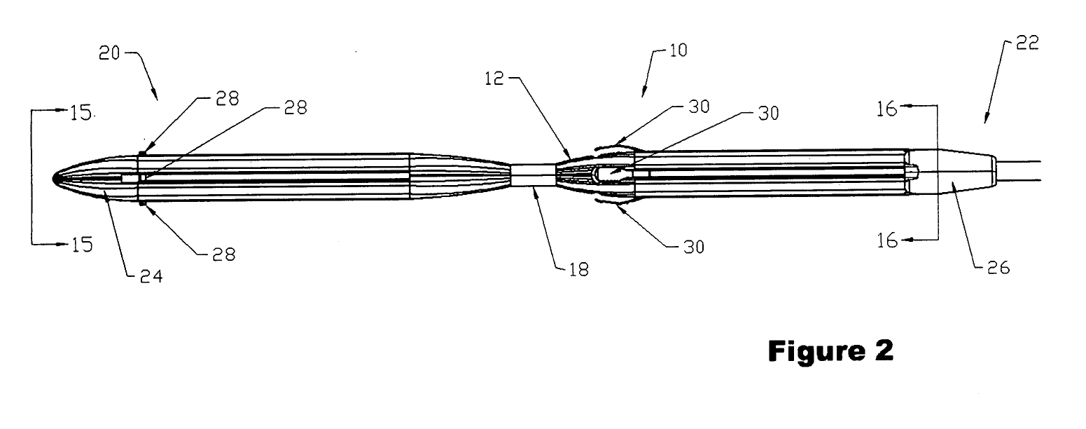

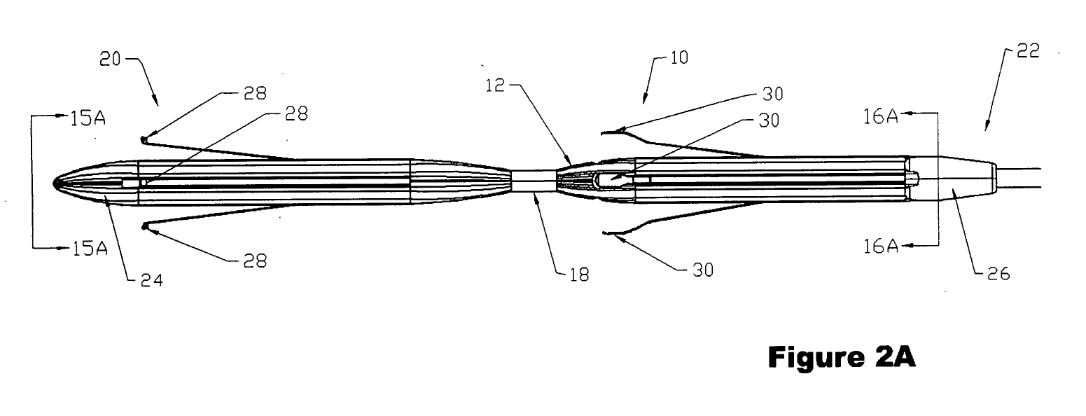

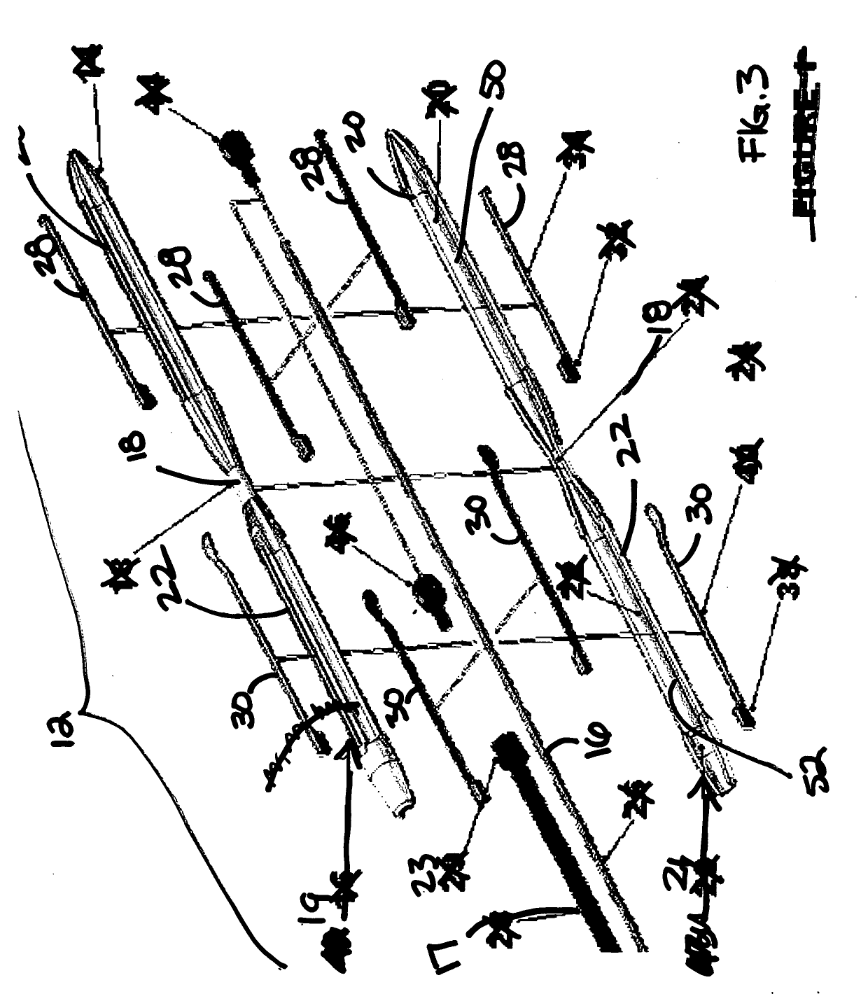

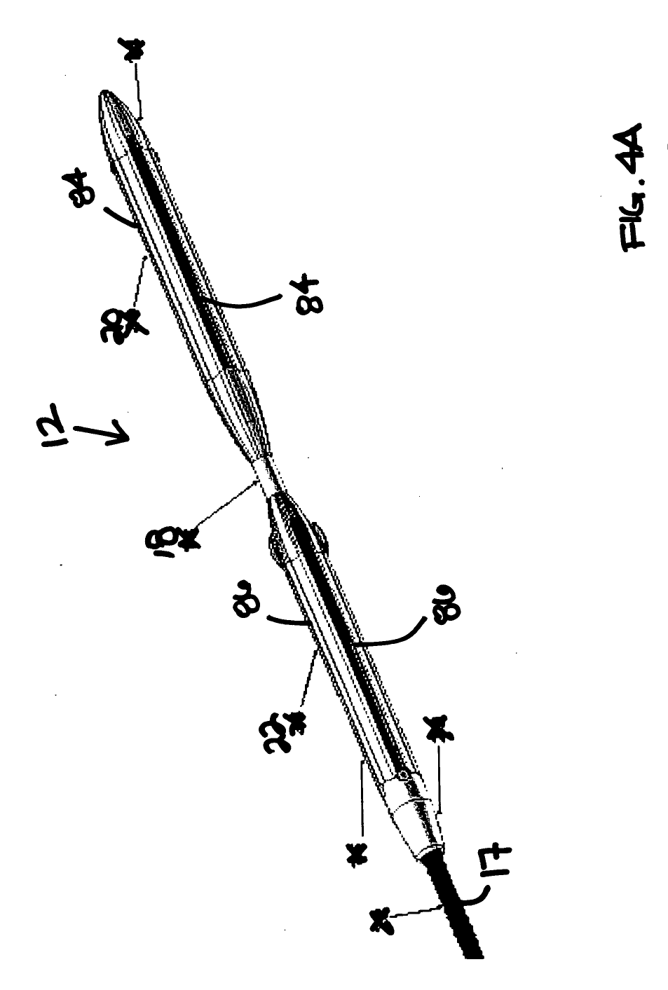

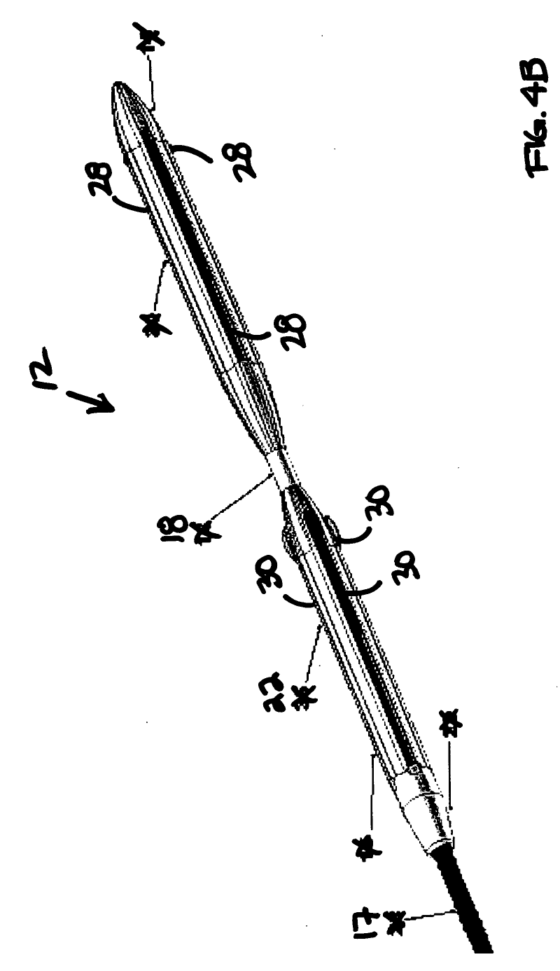

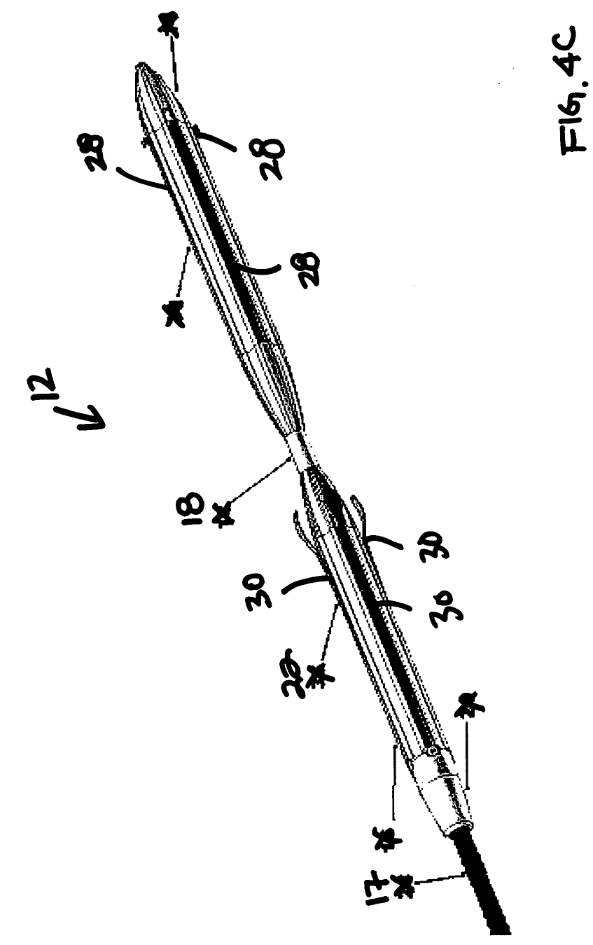

Referring to FIGS. 2, 2A and 3, the bifurcated assembly 12 comprises a hollow conduit member 18 joined between a distal portion 20 having a cutter head 24 including a plurality of retractable, radially oriented cutting blades 28 and proximal portion 22 having a guide head 26 including a plurality of retractable, radially oriented guide fins 30. The distal portion 20 defines a first chamber 50 in communication with a plurality of slots 84 (FIG. 4A) defined around the cutter head 24 adapted to permit each cutting blade 28 to extend radially outward, while the proximal portion defines a second chamber 52 that communicates with the first chamber 50 through a passageway 54 defined by conduit member 18. Similarly, second chamber 52 communicates with a plurality of slots 86 (FIG. 4A) which permit the guide fins 30 to extend radially outward from the guide head 26. The catheter wire 16 is received within the bifurcated assembly 12 at the proximal portion 22 through the first chamber 50 and passageway 54, before terminating inside second chamber 52. In addition, the catheter 17 is operatively associated with a swivel component 23 disposed inside the second chamber 52.

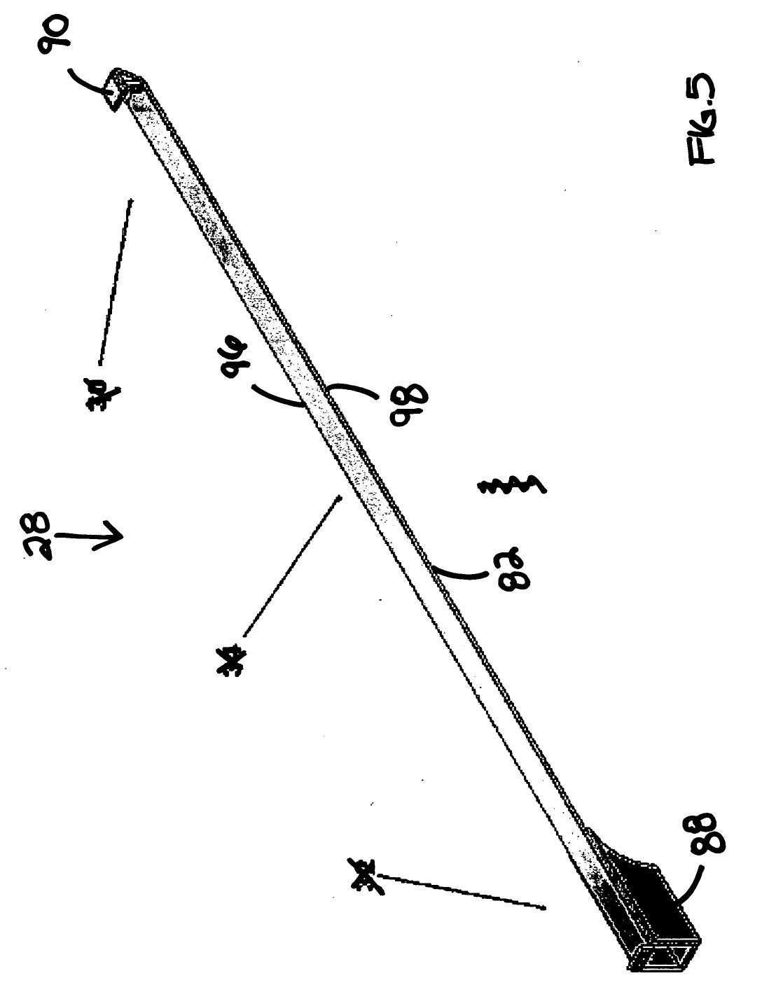

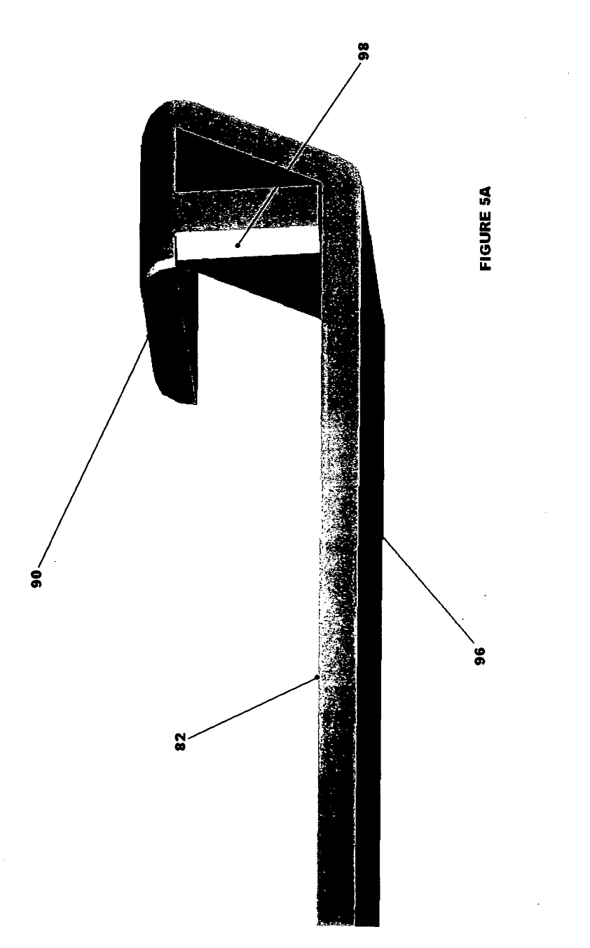

Referring to FIG. 5, each cutting blade 28 comprises a blade body 82 extendable through the slot 84 defined by cutter head 24 and a blade end 80 disposed inside the first chamber 50. The blade end 80 is pivotally attached to a rearward part of the cutter head 24 and defines a channel portion 88, while the blade body 82 terminates in a formed free end 90. As further shown in FIG. 5A, the blade body 82 defines a long flat bottom edge 96, ending in a formed free end 90 where there is a sharp edge 98 that is adapted for cutting valves of a vein.

Referring back to FIG. 3, the guide head 26 preferably includes a set of four guide fins 30 radially disposed equidistantly around the proximal portion 22 with each guide fin 30 having a guide body 94 extending through slot 86 and disposed inside second chamber 50. The guide end 92 is pivotally attached to a rearward part of proximal portion 20. Similar to the cutting blade 28, each end 92 defines a channel portion 89, while the guide body 94 terminates in a formed free end 91.

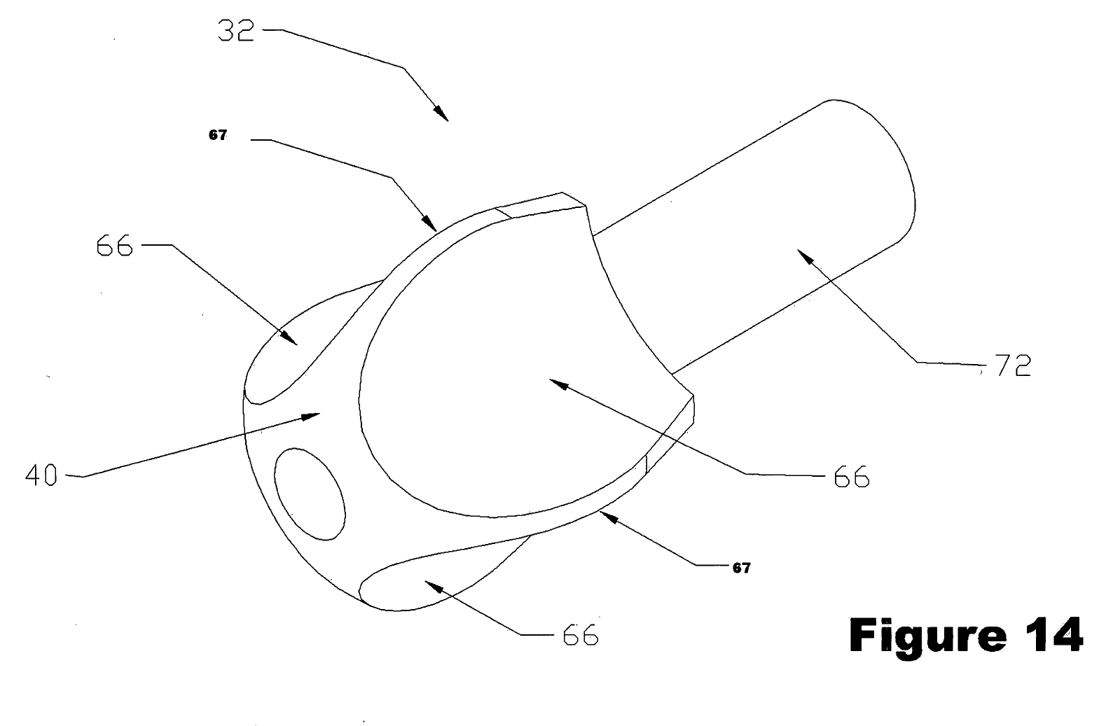

The catheter wire 16 defines a distal terminal end 100 operatively engaged with a first wedge member 32 moveably disposed inside the proximal portion 20. Referring to FIG. 14, the first wedge member 32 comprises a wedge body 40 and a stem 72 with wedge member 32 being movable within the first chamber 50 and adapted to engage the long flat bottom edge 96 of the blade body 82 when the catheter wire 16 is manipulated by handle 14 as shall be discussed in greater detail below. Preferably, the wedge body 40 includes a set of opposing four-sided depression portions 66 and has the general appearance of a Maltese cross, although other suitable configurations that fall within the scope of the present invention are contemplated.

The manipulation of the catheter wire 16 by handle 14 causes the first wedge member 32 to move which engages the long flat bottom edge 96 of each cutting blade 28 with each respective peak 67 of first wedge member 32. Since the first wedge member 32 has a slightly greater diameter than the long flat bottom edge 96 of each cutting blade 28 this structural arrangement permits the wedge member 32 to gradually push each cutting blade 28 outwardly through slot 84 as illustrated in the sequence of FIGS. 4A, 4B and 4C.

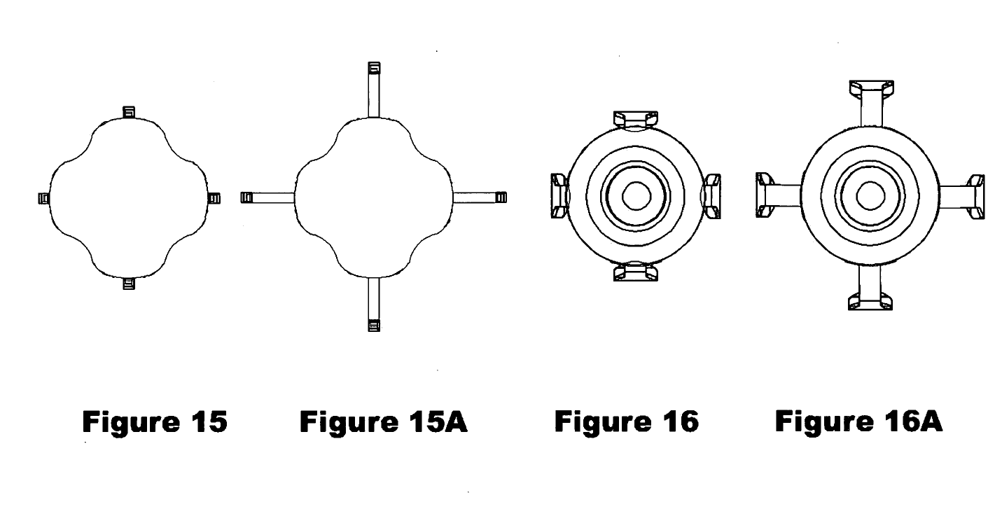

In FIG. 4A, the cutting blades 28 are fully retracted inside first chamber 50, while in FIG. 4B the moving engagement of the first wedge member 32 against each cutting blade 28 causes the blades 28 to extend through each slot 84. As such, the incremental advancement of the first wedge member 32 in relation to each cutting blade 28 effectively changes the degree to which the blade 28 emerges above the surface of the cutter head 24 from first chamber 50 into a cutting position as the first wedge member 28 moves along the long flat bottom edge 96 of each blade 28 as shown in FIG.4C. In operation, each cutting blade 28 acts as a spring so that once the force from the first wedge member 32 is removed each cutting blade 28 returns to the originally fully retracted position inside the first chamber 50 (FIG. 4A). By this structural arrangement, the effective cutting diameter of the cutting blades 28 can be adjusted by the handle 14 through manipulation of the catheter wire 16 in a forward and backward movement with respect to bifurcated assembly 12. As illustrated in FIGS. 15 and 15A, the effective cutting diameter of the cutting blades 28 in the fully retracted position and the fully expanded position are shown. Preferably, the cutter head 24 includes a set of opposing four-sided depression portions 66 and has the general appearance of a Maltese cross, although other suitable configurations that fall within the scope of the present invention are contemplated. FIGS. 16 and 16A illustrate the effective diameter of the guide fins 30 in the fully retracted position and the fully expanded position. Preferably, the guide head 26 includes a set of opposing four-sided depression portions 66 and has the general appearance of a Maltese cross, although other suitable configurations that fall within the scope of the present invention are contemplated.

Similarly, second wedge member 34 is operatively engaged to catheter wire 16 and slidably disposed within second chamber 52 of guide head 26. When the catheter wire 16 is manipulated, the second wedge member 34 is movable in a forward and backward movement inside second chamber 52 and engageable with the inner edge of each guide fin 30. Since the second wedge member 34 has a slightly greater diameter than the inner edge of each guide fin 30 this structural arrangement permits the second wedge member 34 to push the guide fins 30 outwardly through slot 86 as the second wedge member 34 advances along the inner edge of each guide fin 30. The incremental advancement of the second wedge member 34 in relation to the guide body 94 effectively changes the degree to which each guide fin 30 emerges above the surface of the guide head 26 from the second chamber 52. Similar to the operation of the cutter head 24, the guide fins 30 collectively act as a spring so that once the force from the second wedge member 34 is removed by manipulation of the catheter wire 16, the guide fins 30 return to their originally retracted position inside second chamber 52 (FIG. 4A). As such, manipulation of the catheter wire 16 controls the effective diameter of the guide fins 30 as well as function to maintain the bifurcated assembly 12 in a centralized position inside the lumen of the vein during operation of the valvulotome device 10.

Because the first and second wedge members 32 and 34 are centrally disposed within the first and second chambers 50, 52, respectively, the cutting blades 28 and guide fins 30 emerge equidistantly therefrom to ensure the centralized passage through the lumen of the vein of the bifurcated assembly 12 as well as uniform cutting of the venous valves by the cutter body 24, while avoiding damage to the venous luminal walls. As such, the simultaneous adjustability of the cutting blades 28 and guide fins 30 permits the user to adjust the valvulotome device 10 “on-the-fly” when a vein of varying inner diameter is presented during the cutting procedure.

Preferably, the effective diameter of the cutting blades 28 in the fully retracted position is 2.0 mm, while in the fully expanded position the effective cutting diameter is 4.0 mm. This calibration system provides the user of the valvulotome device 10 with the capability to adjust the effective cutting diameter of the cutting blades 28 to a midrange value of 3.0 mm by manipulation of the catheter wire 16. Accordingly, the valvulotome device 10 provides the ability to adjust the effective cutting diameter of the cutter head 24 to 2.0 mm, 3.0 mm and 4.0 mm by the user.

FIG. 7 illustrates the means for enabling the user to control the effective cutting diameter of the cutter head 24. As noted above, manipulation of the catheter wire 16 directly affects simultaneous movement of the first and second wedge members 32, 34, which in turn respectively engage the cutting blades 28 and guide fins 30. Because of this direct relational movement between these various structural components, the valvulotome device 10 can be directly calibrated such that a desired incremental distance of remote movement of the first and second wedge members 32, 34 within the bifurcated assembly 12 can be accurately achieved by the user by effecting the identical, incremental distance movement of the catheter wire 16 at handle 14. In addition, the bifurcated assembly 12 is free to swivel about its axis within the lumen of the vein, as seen in FIG. 6. The catheter 17 has attached to its distal end a swivel component 23. The swivel component 23 consists of a profile change of the catheter 17 to create a flange. The swivel component 23, which is permanently fixed the distal end of the catheter 17, is housed inside the guide head 22. The swivel component 23 remains fixed while the bifurcated assembly 12 can rotate about its axis. The bifurcated assembly 12 is fixed from motion in the longitudinal direction. The catheter wire 16 is free to pass through the swivel component 23 unobstructed and does not rotate.

Referring to FIGS. 7-11, the handle 14 is adapted for use by a single hand of the user for manipulating the catheter wire 16 and adjusting the cutting blades 28 and guide fins 30. The handle 14 comprises a handle body 36 having right housing 56 and left housing 58 that encases a retainer rack 44 and defines an opening 74 having a dial 46 rotatably disposed therethrough. As shown, the dial 46 defines a gripping surface 64 along the periphery thereof adapted for movement by a user's thumb and a pinion 78 adapted for operative engagement to the retainer rack 44 for manipulation of the catheter wire 16. In addition, the dial 46 includes a plurality of visual indicia, such as indicator dots, for providing the user a visual verification of the diameter setting related to the effective cutting diameter of the cutting blades 28. Retainer rack 44 includes a first rack 60 and second rack 62 which are operatively engaged to the proximal end 76 of catheter wire 16. As further shown, catheter 17 is engaged through a relief strain 48 for defining the front portion of handle 14.

To ensure that the cutting blades 28 remain at the desired effective cutting diameter, the handle 14 further includes a locking mechanism 38 having locking notches 102. Legs 104 of the first and second racks 60, 62 are adapted to engage corresponding locking notches 102 to lock the catheter wire 16 in a locked position.

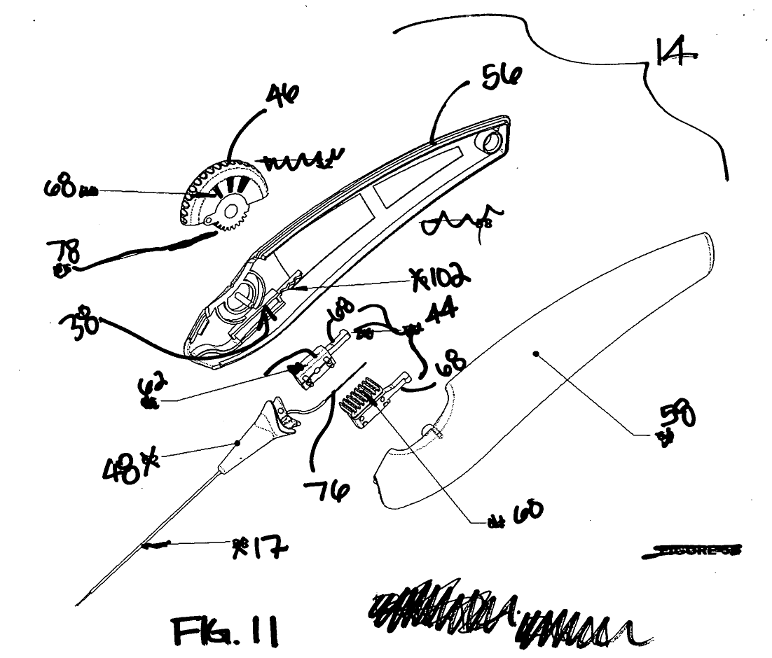

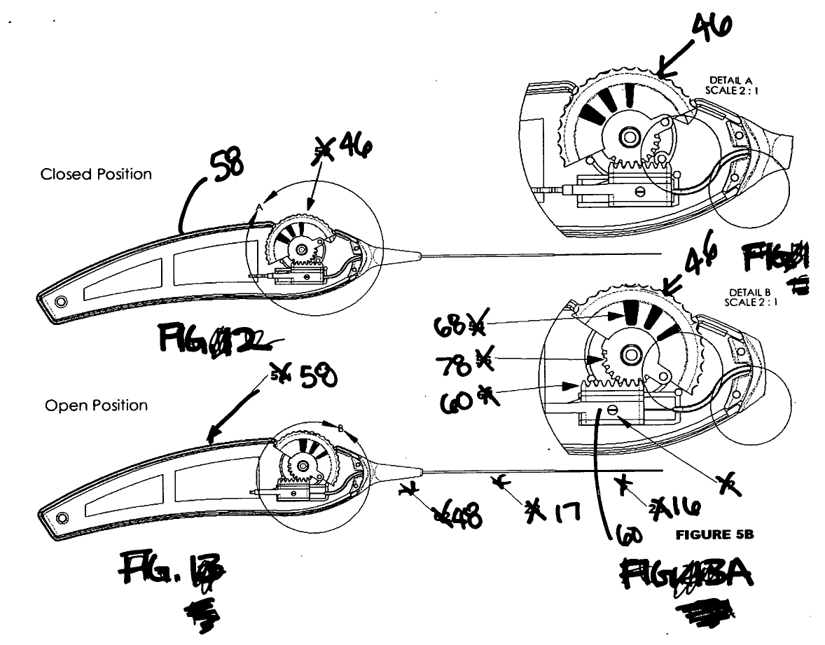

Referring to FIG. 12, the handle 14 is shown in the closed position for locking the catheter wire 16 in place. In the closed position, the pinion 78 is engaged to one portion of retainer rack 44 (FIG. 12A). In the closed position shown in FIG. 13, the pinion 78 is engaged to another portion of retainer rack 44 (FIG. 13A).

It should be understood from the foregoing that, while particular embodiments of the invention have been illustrated and described, various modifications can be made thereto without departing from the spirit and scope of the invention as will be apparent to those skilled in the art. Such changes and modifications are within the scope and teaching of this invention as defined in the claims appended hereto.

1. A valvulotome device comprising:
a handle;
a bifurcated assembly operatively engaged to said handle having a distal portion including a cutter head, said cutter head having a plurality of adjustable cutting blades and a proximal portion including a guide head, said guide head having a plurality of adjustable guide fins;
a catheter wire operatively engaged to said cutter head and said guide head through said handle and disposed within said bifurcated assembly, wherein said catheter wire permits remote manipulation of said cutter head and said guide head; and
first and second wedge members operatively engaged to said catheter wire and movably disposed inside said cutter head and said guide head, respectively, and in further operative engagement with said plurality of adjustable cutting blades and said plurality of adjustable guide fins, such that movement of said first and second wedge members controls the movement of said plurality of adjustable cutting blades and said plurality of adjustable guide fins, respectively.

2. The device of claim 1, wherein said cutter head and said guide head each define first and second chambers adapted to slidably receive said first and second wedge members, respectively.

3. The device of claim 2, wherein said first and second wedge members are centrally disposed respectively for simultaneous forward and backward movement in said first chamber of said cutter head and in said second chamber of said guide head, respectively.

4. The device of claim 2, wherein said plurality of adjustable cutting blades are retractable or extendable through a corresponding slot in communication with said first chamber.

5. The device of claim 4, wherein said plurality of adjustable cutting blades each comprise a blade body and a blade end, said blade end being pivotally attached to a rearward part of said cutter head and said blade body extending through a corresponding slot and terminating in a formed free end thereof.

6. The device of claim 1, wherein said plurality of adjustable cutting blades are radially oriented.

7. The device of claim 4, wherein said plurality of adjustable cutting blades are operable to retract into and extend from said first chamber through said corresponding slot.

8. The device of claim 1, wherein said plurality of adjustable cutting blades are pivotally attached at a proximal end of said cutter head.

9. The device of claim 2, wherein said plurality of adjustable guide fins are retractable or extendable within a corresponding slot in communication with said second chamber of said guide head.

10. The device of claim 9, wherein said plurality of adjustable guide fins each comprise a guide body and a guide end, said guide end pivotally attached to a rearward part of said guide head and said guide body extending through a corresponding slot and terminating in a formed free end thereof.

11. The device of claim 1, wherein said plurality of adjustable guide fins are radially oriented.

12. The device of claim 2, wherein said plurality of adjustable guide fins are operable to retract into and extend from said second chamber of said guide head.

13. The device of claim 1, wherein said plurality of adjustable guide fins are pivotally attached at a proximal end of said guide head.

14. The device of claim 1, wherein said remote manipulation of said catheter wire causes said cutting blades and said guide fins to retract or extend.

15. The device of claim 1, wherein said first and second wedge members comprise a wedge body and a stem.

16. The device of claim 15, wherein said wedge body comprises a set of four-sided opposing depression portions.

17. The device of claim 16, wherein said first wedge member has a slightly greater diameter than a flat inner edge of each cutting blade and said second wedge member has a slightly greater diameter than an inner edge of each guide fin such that upon manipulation of said catheter wire by said handle, said first wedge member moves to engage said flat inner edge of each cutting blade with each respective depression portion of said first wedge member to incrementally engage each cutting blade outwardly through said cutter head as said second wedge member incrementally engages each guide fin to push said guide fins outwardly through said guide head.

18. The device of claim 1, wherein said handle further comprises a calibration system disposed on said handle and operatively associated with said catheter wire to control the extension and retraction of said plurality of retractable cutting blades from said cutter head and said plurality of retractable guide fins from said guide head.

19. The device of claim 1, wherein said handle comprises
a handle body,
a retainer rack encased by said handle body and defining an opening, and
a dial operatively associated with said retainer rack and rotatably disposed through said opening.

20. The device of claim 19 wherein said retainer rack includes a first rack and a second rack that are operatively engaged to a proximal end of said catheter wire.

21. The device of claim 19 further comprising a locking mechanism comprising a plurality of locking notches, wherein a plurality of legs of said first rack and said second rack are adapted to engage to said corresponding plurality of locking notches to lock said catheter wire in a locked position.

22. The device of claim 1, further comprising a catheter, said catheter encasing said catheter wire and operatively engaged to a swivel component such that said bifurcated assembly may rotate about the axis of said bifurcated assembly.

23. A method of using a valvulotome device to cut one or more valves of a vein, the method comprising:
a) providing a valvulotome device comprising a handle operatively engaged to a bifurcated assembly having a distal portion including a cutter head said cutter head having a plurality of retractable, radially oriented cutting blades and a proximal portion including a guide head said guide head having a plurality of retractable, radially oriented guide fins, wherein said catheter wire is operatively engaged to first and second wedge members disposed inside said cutter head and guide head, respectively, and in operative engagement with said plurality of cutting blades and plurality of guide fins such that movement of each first and second wedge members controls said cutting diameter said plurality of cutting blades and said effective diameter of said guide fins, respectively;
b) inserting said bifurcated body into said vein; and
c) pulling said bifurcated body back through said vein such that said one or more valves of said vein are cut.

24. The method of claim 23, further comprising manipulating said catheter wire in a forward and backward movement to control said effective diameter of said plurality of guide fins and said plurality of cutting blades.

25. The method of claim 23, further comprising manipulating said catheter wire in a forward and backward movement with respect to said bifurcated assembly to adjust said effective diameter of said plurality of guide fins.

26. The method of claim 23, further comprising manipulating said catheter wire to control said effective diameter of said plurality of guide fins to maintain said bifurcated assembly in a centralized position inside said lumen of said vein during operation of said vavlulotome device.

27. A bifurcated assembly for use in a valvelotome device comprising:
a conduit member joined between a distal portion having a cutter head defining a chamber and a proximal portion having a guide head defining a chamber said head including a plurality of retractable radially oriented cutting blades, and said guide head including a plurality of retractable, radially oriented guide fins,
wherein each of said cutter head and said guide head includes a wedge member disposed in each respective chamber and being adapted to operatively engage a catheter wire such that the diameters of cutting blades and guide fins can be adjusted by manipulation of said catheter wire.

28. The assembly of claim 27, wherein said plurality of adjustable cutting blades and said plurality of adjustable guide fins are retractable or extendable through a corresponding slot in communication with each respective chamber.

29. A valvulotome device comprising:
the bifurcated assembly comprising cutting means for cutting valves in a vein of a patient and guide means for maintaining said cutting means in a continual centralizing, self-alignment within a lumen of a vein, and
means engaged to said bifurcated assembly to permit remote manipulation and navigation of said valvulotome device by a user through said vein of a patient.
